(19) United States
(12) Patent Application Publication
(10) Pub. No.: US 2018/0333046 A1
(43) Pub. Date: Nov. 22, 2018

(54) ENDOSCOPIC CAMERA CONTROL SYSTEM

(71) Applicant: Mehringer-Pieper Innovations, LLC, Jasper, IN (US)

(72) Inventors: Noah J. Mehringer, Jasper, IN (US); Nicholas J. Pieper, Jasper, IN (US)

(21) Appl. No.: 15/977,652

(22) Filed: May 11, 2018

Related U.S. Application Data
(60) Provisional application No. 62/506,967, filed on May 16, 2017.

(51) Int. Cl. A61B 1/045; A61B 1/00; H04N 5/225; A61B 1/04

(52) U.S. Cl. CPC A61B 1/045; A61B 1/00147; A61B 1/00133; H04N 5/2252; H04N 2005/2255; A61B 1/042; A61B 1/00045; A61B 1/00016; A61B 1/00188

(57) ABSTRACT

A camera control system includes an orientation control unit attached to a headset and a camera attached to an endoscope. In response to rotation of the headset, the orientation control unit transmits a control signal to a control unit attached to the support. The control unit activates servomotors to adjust the orientation of the camera on two axes, corresponding to rotation of the headset. Video signals captured by the camera are relayed to a user wearing the headset.

This application claims the benefit of U.S. provisional patent application Ser. No. 62/506,967, filed May 16, 2017, for CAMERA CONTROL SYSTEM, incorporated herein by reference.

### FIELD OF THE INVENTION

A camera control system includes an orientation control unit attached to a headset and a camera attached to an endoscope. In response to rotation of the headset, the orientation control unit transmits a control signal to a control unit attached to the support. The control unit activates servomotors to adjust the orientation of the camera on two axes, corresponding to rotation of the headset. Video signals captured by the camera are relayed to a user wearing the headset.

### BACKGROUND OF THE INVENTION

Over the years a variety of minimally invasive robotic systems have been developed to increase surgical dexterity as well as to minimize the size of the incision and consequent physical trauma suffered by the patient. However, these robotic systems cannot eliminate user error, as a surgeon manipulating robotic grippers may, for example, drop a surgical device or biological object inside of the patient's body cavity during an operation. Current robotic surgical systems typically view the inside of an abdominal body cavity via a camera rigidly mounted and limited in maneuverability. Thus, dropping objects due to user or mechanical error can create complications when the surgeon is not able to view the dropped object within the camera's visual field. The camera can oftentimes be manipulated manually to change the field of vision, but this action is limited by the elasticity of the patient's skin surrounding the incision through which the camera is inserted—and by the flexibility of the member supporting the camera. In some robotic surgery systems, a camera may be mounted on or in a flexible tube to provide a modicum of maneuverability. However, the maneuverability of the camera and resulting capability to change the field of vision is limited by the flexibility of the tube. As a result, dropped objects can be cause for serious complications or delays in operation time. Sizeable delays in operation time can have negative results on the patient, who must remain anaesthetized for a longer period of time, and may increase the risk of surgery-related injuries.

It was realized by the inventors that improvements in controlling cameras used with robotic surgery were needed to address these challenges and provide other important advantages.

### SUMMARY

The disclosed system and method for controlling a moveable camera address these challenges and provide multiple benefits to a user. More specifically, the disclosed system and method provide a user with an increased field of vision and range of camera motion as compared to a camera fixedly mounted on a support. The disclosed system and method also provide the user with a means for controlling a camera while leaving the user's hands and feet free.

In some embodiments, a camera control system comprises a headset configured to be worn by a user, the headset including an orientation detection unit configured to transmit a control signal in response to detecting an orientation of the orientation detection unit; and an endoscope including a camera, means for rotating the camera on a first axis, means for rotating the camera on a second axis, and a control unit configured to receive the control signal and control the means for rotating the camera on the first axis and the means for rotating the camera on the second axis. In further embodiments, the first axis and the second axis are not identical. In certain embodiments, a video transmitter is in communication with the camera, wherein a video signal is transmitted from the camera to the video transmitter and from the video transmitter to the headset. In some embodiments, the headset includes means for displaying the video signal to the user. In further embodiments, the orientation detection unit includes a gyroscope and a control signal transmitter for transmitting the control signal to the control unit. In certain embodiments, the orientation detection unit detects the orientation of the orientation detection unit on the first axis and the second axis, and wherein the control unit rotates the camera on the first axis and the second axis to match the orientation of the orientation detection unit. In some embodiments, the orientation detection unit detects the orientation of the orientation detection unit on a third axis, and wherein the control unit controls a zoom feature of the camera based on the orientation of the detection unit on the third axis. In further embodiments, the control unit is configured to control the means for rotating the camera on the first axis and the means for rotating the camera on the second axis when the control signal exceeds a predetermined rotation threshold. In certain embodiments, the predetermined rotation threshold is one degree, two degrees, three degrees, four degrees, or five degrees of rotation in one of the first axis and the second axis. In some embodiments, the camera control system includes a haptic feedback system, wherein the haptic feedback system is activated when the control signal exceeds the predetermined rotation threshold. In further embodiments, the haptic feedback system includes at least one vibrating component attached to the headset. In certain embodiments, the endoscope includes a flexible housing, and wherein the camera located at least partially within the flexible housing. In some embodiments, the means for rotating the camera on the first axis is a first servomotor coupled to one of the flexible housing and the camera, whereby activation of the first servomotor deflects the camera along the first axis. In further embodiments, the means for rotating the camera on the second axis is a second servomotor coupled to one of the flexible housing and the camera, wherein activation of the second servomotor deflects the camera along the second axis. In certain embodiments, the camera control system includes at least one pressure sensor mounted on the flexible housing. In some embodiments, the at least one pressure sensor is configured to output a pressure signal to the control unit. In further embodiments, the control unit controls the means for rotating the camera on the first axis and the means for rotating the camera on the second axis based on the control signal and the pressure signal. In certain embodiments, a haptic feedback system, wherein the haptic feedback system is activated when the pressure signal exceeds a predetermined pressure threshold. In some embodiments, the haptic feedback system includes at least one vibrating component attached to the headset.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein is not necessarily intended to address every object described herein, and each embodiment does not necessarily include each feature described. Other forms, embodiments, objects, advantages, benefits, features, and aspects of the present invention will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1A depicts a front view of a user wearing an embodiment of a headset.

FIG. 1B depicts a top perspective view of the user and headset of FIG. 1A.

FIG. 1C depicts a side view of the user and headset of FIG. 1A.

FIG. 2A depicts a perspective view of an embodiment of an endoscope.

FIG. 2B depicts a top view of the endoscope of FIG. 2A.

FIG. 3A depicts a top view of the control housing.

FIG. 3B depicts a top perspective view of the control housing of FIG. 3A.

FIG. 4A depicts a top view of the actuator housing.

FIG. 4B depicts a top perspective view of the actuator housing of FIG. 4A.

FIG. 5 depicts a side perspective view of a flexible housing.

### DETAILED DESCRIPTION OF THE PREFERRED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages, or may include different advantages. Any advantages described herein are not to be construed as limiting to any of the claims.

Specific quantities (spatial dimensions, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

The disclosed endoscopic camera control system includes an endoscope and a wearable headset. The headset uses head tracking with a gyroscope to allow the movement of a user's head to be reflected in the movements of a camera positioned in a flexible housing at the end of the endoscope, which may be inserted within a body cavity. Video from the camera is transmitted to the headset, providing the user with a real-time first person view from inside the body cavity. The flexibility of the camera's housing, as well as the mechanisms for flexing it, allow the user to manipulate the camera in two degrees of freedom simply by moving the user's head, leaving the user's hands and feet free.

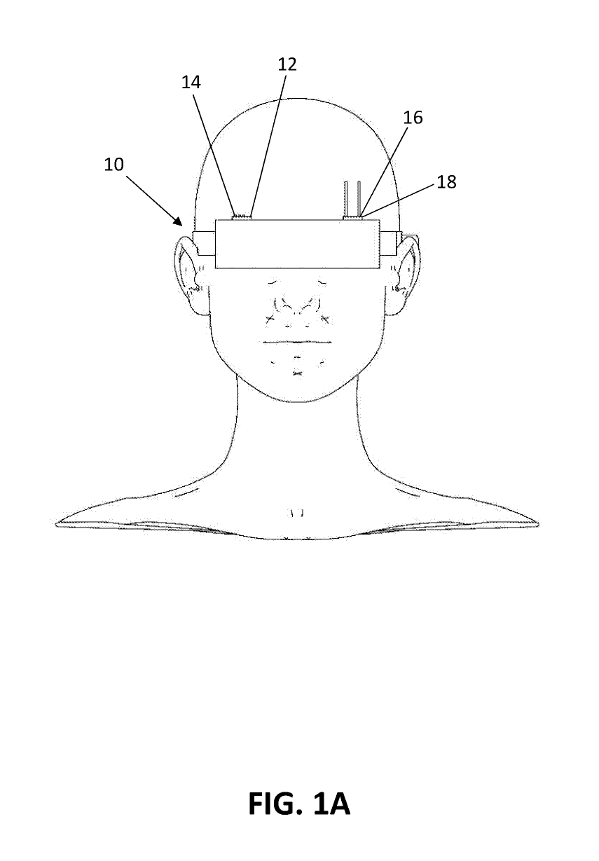

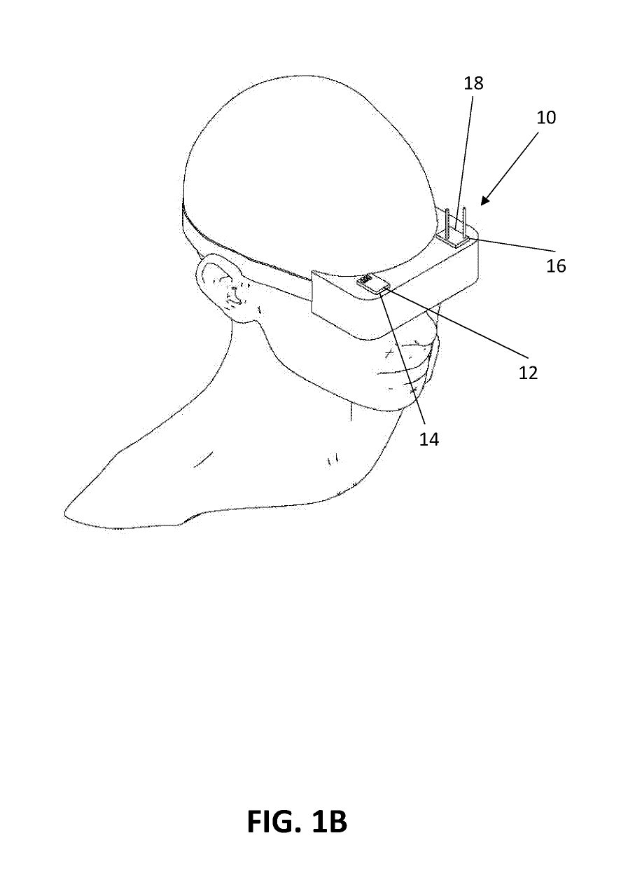

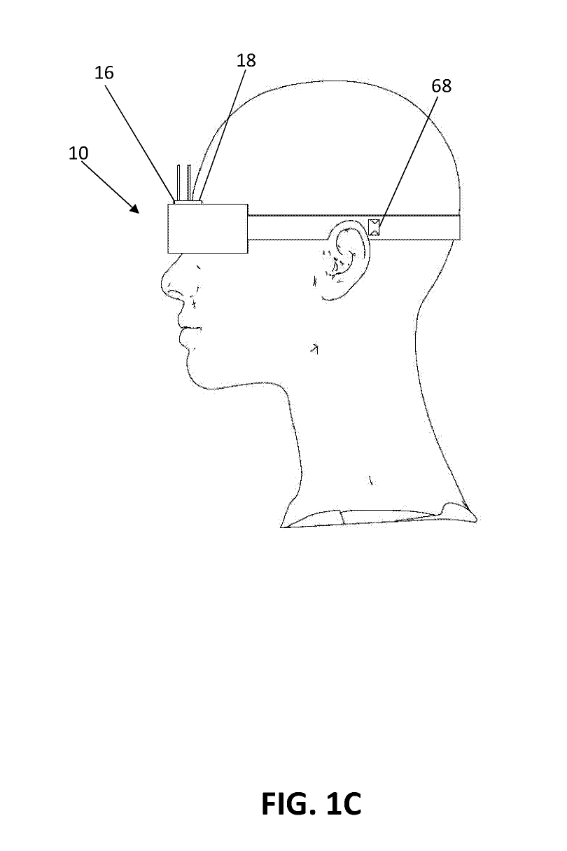

Referring to FIGS. 1A-1C, the headset 10 is a first-person view (“FPV”) headset 10 including an orientation detection unit (“ODU”) 12. The ODU 12 includes a means for detecting and outputting the orientation of the ODU 12, such as one or more gyroscopes 14, such as a MEMS gyroscope, and a control signal transmitter 16. The control signal transmitter 16 is configured to transmit digital orientation data from the gyroscopes 14 or other means for detecting and outputting the orientation of the ODU 12, the transmitted orientation data being referred to as the control signal. In some embodiments, a single gyroscope 14 is integral to the headset 10, centered on the user's midline, and horizontal to the ground when the headset is held level. In other embodiments, as most easily seen in FIG. 1A, the gyroscope 14 may be laterally offset from the user's midline.

The headset 10 further includes a video receiver 18 in communication with the FPV headset 10, such that received video signals are displayed to the user wearing the headset 10. In preferred embodiments, the FPV headset 10 allows for stereoscopic vision. In some embodiments, the control signal transmitter 16 and video receiver 18 are embodied in a single transceiver or transmitter-receiver 16, 18.

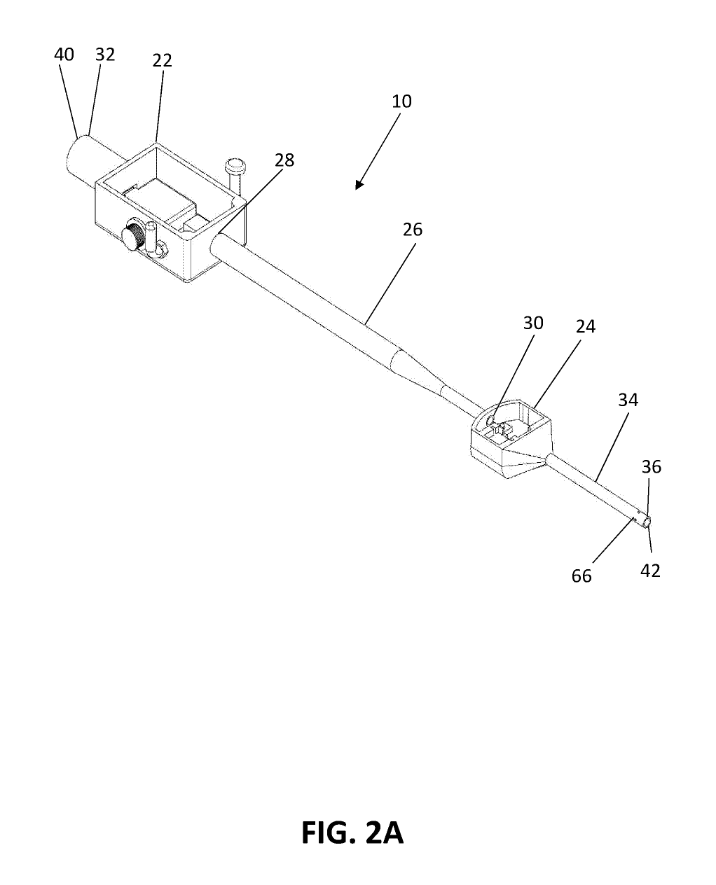

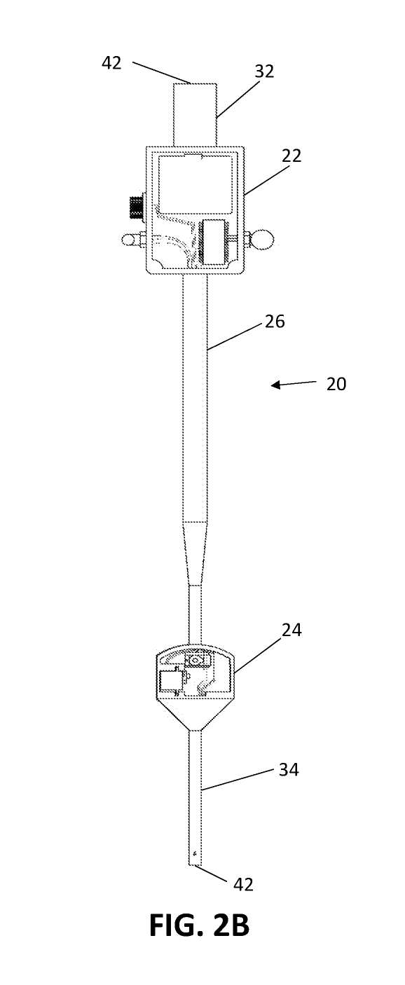

Referring to FIGS. 2A-5, an embodiment of the endoscope 20 includes a control housing 22 attached to an actuator housing 24 via an elongated, rigid cylindrical support member 26. The control housing 22 and actuator housing 24 each include portholes 28, 30, and the support member 26 extends into each porthole 28, 30 to mechanically connect the control housing 22 to the actuator housing 24. Extending from the control housing 22 opposite the support member 26 is an optional mounting fixture 32. Extending from the actuator housing 24 opposite the elongated support 26 is an elongated camera mount 34. In this embodiment, the elongated camera mount 34 is a hollow tube including an opening 36 adapted to receive a cylindrical flexible housing 38. For the purposes of orientation, the proximal end 40 of the endoscope 20 is the end of the mounting fixture 32, furthest from the camera mount 34. The distal end 42 of the endoscope 20 is the end of the camera mount 34, furthest from the mounting fixture 32. A camera 44 and, optionally, a light source 46, are located at least partially within the flexible housing 38. When in use in a surgical procedure, the distal end 42 of the endoscope 20 is closest to, or positioned at least partially inside, a body cavity.

In some embodiments, the endoscope 20 may be supported by a standing rig, as generally known in the art, which engages at least one of the mounting fixture 32, the control housing 22, the support 26, and the actuator housing 24. In other embodiments, the endoscope 20 may lack the mounting fixture 32 and may be handheld.

The control housing 22 includes a microcontroller 48, video transmitter 50, digital receiver 52 and power supply 54. The digital receiver 52 is at least partially contained within the control housing 22 and is configured to receive the control signal. In some embodiments, the digital receiver 52 continuously receives the control signal while both the microcontroller 48 and ODU 12 are powered. The microcontroller 48 is at least partially contained within the control housing 22 and is in digital communication with the digital receiver 52. In embodiments wherein the ODU 22 includes multiple gyroscopes 48, measuring different or similar axial position values, the microcontroller 48 may receive and compile the different orientation data present in the control signal to minimize errors from any single gyroscope 14 using techniques commonly known in the art.

The actuator housing 24 includes means for rotating the camera on a first axis and means for rotating the camera on a second axis. In some embodiments, the means for rotating the camera on a first axis is a first servomotor 56 and the means for rotating the camera on a second axis is a second servomotor 58.

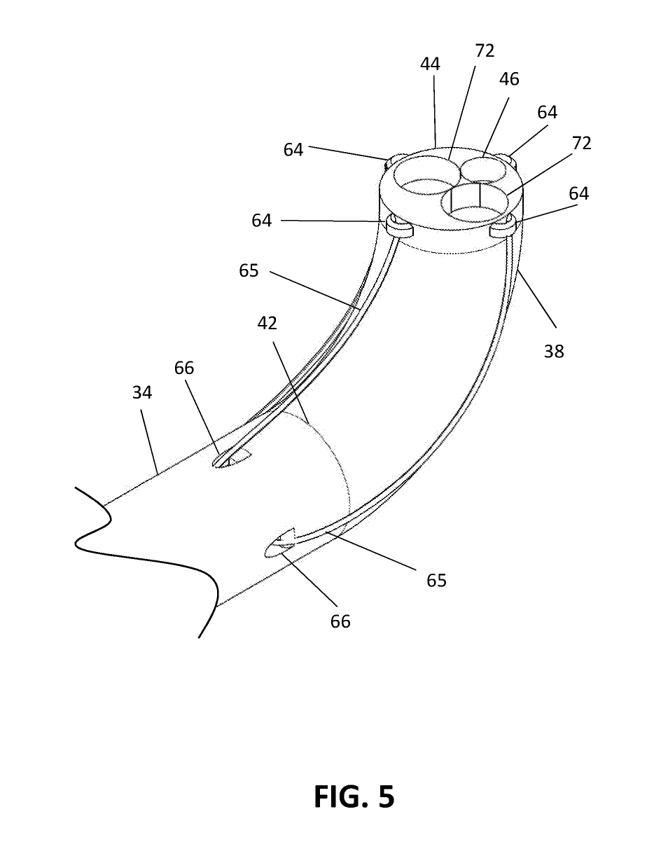

Referring to FIG. 5, the disclosed endoscopic camera control system includes a camera 44 mounted at least partially within in a flexible housing 38. The flexible housing 38 may be a single flexible cylindrical member as shown in FIG. 5, or may be a segmented, rigid structure separated into segments by one or more movable joints. This includes the use of “ribbing” to allow the structure to bend in two degrees of freedom. The flexible housing 38 is orientable with at least two degrees of freedom, thereby allowing the camera 44 to be rotated on a first axis and on a second axis.

In some embodiments, the camera 44 has a diameter of less than 3 cm, less than 1.5 cm, or less than 1 cm. In further embodiments, the camera 44 has a diameter of less than 100 mm, less than 20 mm, about 12 mm, or about 8 mm. In preferred embodiments, the camera 44 is stereoscopic to provide the user with 3D vision on the display of the FPV headset 10. The camera 44 collects images and transmits video signals proximally along the endoscope 20 via a first electrical pathway 60 to the video transmitter 50 in the control housing 22. The video transmitter 46 subsequently transmits the video signal to the video receiver 18 on the FPV headset 10, so that the user may view the images collected from the camera 44 substantially continuously and in real time.

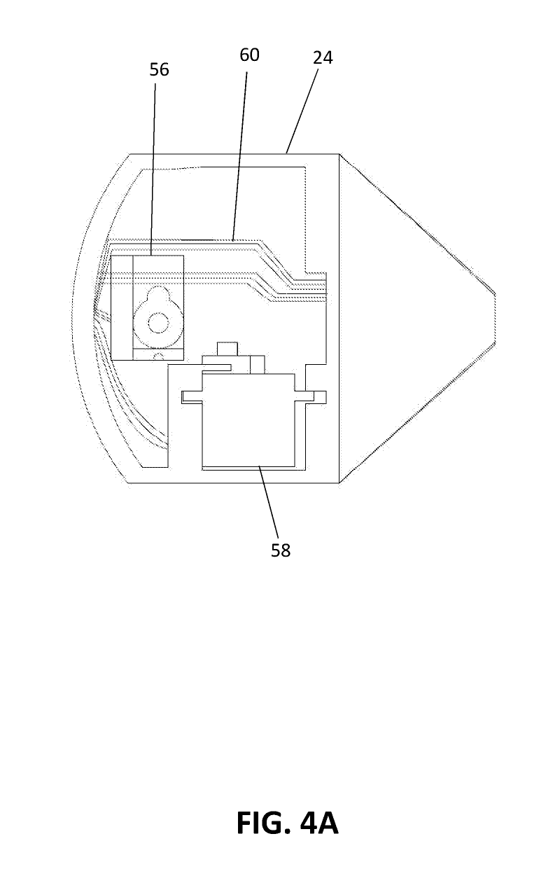

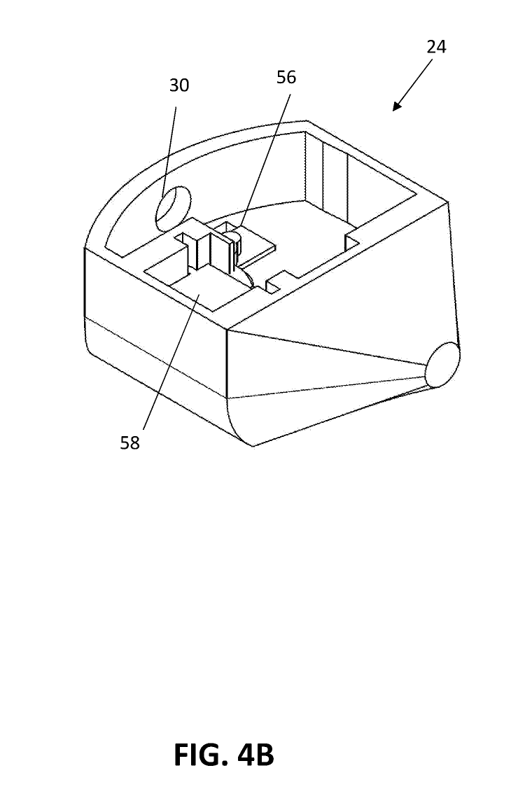

The pre-programmed microcontroller 48 uses orientation data in the control signal to output an electrical signal along a second electrical pathway 62 from the control housing 22 to the first and second servomotors 56, 58 in the actuator housing 24. In some embodiments, the second electrical pathway 62 is at least one wire extending along or through the elongated support member 26, which may be a hollow tube. The servomotors 56, 58, when powered, transfer mechanical energy distally down the endoscope 20 to attachment points 64 on the camera 44 or the flexible housing 38. In some embodiments, the mechanical energy is transferred by semi-rigid wires 65 (omitted from FIG. 4B for clarity, visible in FIG. 5) extending from the servomotors 56, 58, through the interior of camera mount 34, external to the camera mount 34 through apertures 66 in the camera mount 34, and along the exterior of the camera mount 34 and flexible housing 38 to the attachment points 64. The first servomotor 56 applies a pushing or pulling force to one or more wires 65 extending to the camera 44 or the flexible housing 38, thereby exerting a force to deflect the camera 44 along a first axis. The second servomotor 58 applies a pushing or pulling force to one or more wires 65 extending to the camera 44 or the flexible housing 38, thereby exerting a force to deflect the camera 44 along a second axis. In some embodiments, the first axis and second axis are oriented 90 degrees from each other, such that the first axis is a horizontal axis and the second axis is a vertical axis.

In some embodiments, two wires 65 are attached to each of the first and second servomotors 56, 58, and the camera 44 or flexible housing 38 include four attachment points 64 equally spaced about their perimeter. Two wires 65 extend from the first servomotor 56 to attachment points 64 on the left and right sides of the camera 44 or flexible housing 38, and two wires 65 extend from the second servomotor 58 to attachment points 64 on the top and bottom of the camera 44 or flexible housing 38. In alternative embodiments, greater or fewer numbers of wires 65 and attachment points 64 may be used, or the semi-rigid wires 65 may be replaced by other means for mechanically transferring force over a distance. The wires 65 may run along the internal or external surface of the flexible housing 38, and along the internal or external surface of the camera mount 34. In some embodiments, where the wires 65 run along the external surface of the flexible housing 38, the wires and attachment points 64 may be covered by a protective coating to reduce exposure to bodily fluids. In certain embodiments, optional wire guides are mounted on the flexible housing 38 to hold the wires 65 in position.

In use, the microcontroller software calibrates based upon the initial orientation of the headset 10 upon the camera control system being turned on and the software's initial execution. A button or other input may also be implemented to allow the user to reset the initial orientation to the current orientation held when the button is pressed and the software executed. A rotation threshold will be stablished for each degree of freedom. The rotation threshold values specify a range for each degree of freedom within which the user's head orientation will have no effect on the manipulation of the camera 44. In some embodiments, the rotation threshold is ten degrees, five degrees, three degrees or two degrees. Rotation of the user's head—as measured by the ODU 12—of less than the threshold will be ignored by the microcontroller 48 and will not result in movement of the camera 44. The rotation threshold would be predetermined before the operation to the comfort of the surgeon to allow for negligible head movement.

The control signal is transmitted from the ODU 12 to the microcontroller 48, which uses inputs from the gyroscope 14 and preprogrammed software to control the activation of the first and second servomotors 56, 58. When the microcontroller 48 determines that rotation of the user's head along any of the axes are outside the predetermined rotation threshold, the microcontroller 48 activates the corresponding servomotor 56, 58 to deflect the camera 44 to correspond to the rotation of the user's head. For example, if the user's head rotates twenty degrees about the first axis, surpassing a rotation threshold of five degrees, the microcontroller 48 will activate the first servomotor 56 and rotate the camera 44 vertically along the first axis by twenty degrees. In other embodiments, the rotation threshold may be subtracted from the resulting rotation of the camera 44, such that a rotation of the user's head by twenty degrees about the first axis, surpassing a rotation threshold of five degrees, results in a rotation of the camera 44 by fifteen degrees along the first axis. Rotation of the user's head about the first axis by three degrees does not exceed the rotation threshold of five degrees, resulting in no movement of the camera 44. This method of adjusting the rotation of the camera 44 to directly match the rotation of the ODU 12 from the neutral position is referred to as “head tracking” mode.

In some embodiments, the control system includes a means for resetting the camera 44 to a neutral position aligned with the camera mount 34. In certain embodiments, the control system includes a means for switching the camera adjustment mode between “head tracking” and “joystick” modes. “Joystick” mode refers to the utilization of the gyroscope readings based upon the original values in the neutral position; when the user moves his or her head from the original position the servos move in a similar direction until the user returns to original position or the servo is at a maximum value. This “joystick” control method permits the servomotors 56, 58 to be activated at a rate proportional to the extent by which current orientation of the user's head is outside of the rotation threshold of the user's original head position, such that a greater rotation of the user's head in a given direction results in increased rotation speed of the camera 44 in the same direction.

The control signal transmitter 16 may transmit the control signal to the digital receiver 52 continuously or periodically. In some embodiments, the user may pause the transmission to allow the user to readjust the headset 10, take a break, or perform another activity without affecting the orientation of the camera 44. In some embodiments, the headset 10 may include a button, toggle, switch, or similar device in communication with the control signal transmitter 16 configured to prevent the transmission of the control signal. In other embodiments, the endoscope 20 may include a button, toggle, switch, or similar device in communication with the digital receiver 52 configured to prevent the receipt of the control signal by the digital receiver 52.

Optionally, the camera control system may utilize a third degree of freedom about a third axis, the Z axis of the user's head, which passes orthogonally through the plane dividing the ventral and dorsal regions of the user's head. If position values about the third axis of the user's head surpass the rotation threshold from the resting position of the user's head, the gyroscope 14 outputs from the ODU 12 will be interpreted by the microcontroller 48 to manipulate the camera 44 to zoom in or out, provided that the camera 44 is capable of zooming functions. Rotating the user's head about an arbitrary ‘positive’ direction on the third axis will cause the camera 44 to zoom in, while rotating the opposite, ‘negative’ direction would cause the camera 44 to zoom out.

In some embodiments, the camera control system includes a haptic feedback system. The haptic feedback system includes at least one vibrating component 68, such as, for example, an eccentric rotating mass vibration motor or a linear resonant actuator, wherein vibration of the vibrating component 68 can be detected by the user. In some embodiments, as shown in FIG. 1C, the haptic feedback system includes at least one vibrating component 68 attached to the headset 10. In other embodiments, the vibrating component 68 may the worn by or attached to the user separate from the headset 10.

In one embodiment, the at least one vibrating component 68 is activated based on the orientation of the user's head, as detected by the ODU 12. When the user's head rotates sufficiently to exceed the rotation threshold, the at least one vibrating component 68 is activated. The component may vibrate at a low frequency and low intensity. As the user's head increasingly exceeds the rotation threshold, the frequency or intensity of the vibration of the component 68 increases proportionally. The component 68 ceases to vibrate when the user's head returns to the original position. In this embodiment, the haptic feedback system provides the user with tactical feedback confirming that the camera control system is active and the camera 44 is being redirected.

In another embodiment, the at least one vibrating component 68 is activated based on pressure exerted on the flexible housing 38. At least one pressure sensor is mounted on the flexible housing 38, the pressure sensor being in communication with microcontroller 48 via the second electrical pathway 62. The pressure sensor may be mounted on the exterior or, as in the depicted embodiment, interior of the flexible housing 38. The at least one pressure sensor may transmit a pressure signal to the microcontroller 48 continuously, or when pressure is exerted on the pressure sensor. The microcontroller 48 is programmed with a predetermined pressure threshold. Upon detection of a pressure signal exceeding the pressure threshold, the microcontroller 48 may decrease the responsiveness of the servomotors 56, 58 to the control signal (e.g., a head rotation of twenty degrees may result in a camera 44 rotation of ten degrees instead of twenty) or the microcontroller 48 may prevent further rotation of the camera 44. In certain embodiments, the flexible housing 38 includes two pressure sensors oriented on the first axis on opposite sides of the flexible housing 38, and two pressure sensors oriented on the second axis on opposite sides of the flexible housing 38. Upon detection of a pressure signal from a specific pressure sensor exceeding the pressure threshold, the microcontroller 48 prevents further rotation of the camera 44 in the direction of that pressure sensor. The microcontroller 48 may also activate the at least one vibrating component 68 when the pressure signal exceeds the pressure threshold. In this embodiment, the haptic feedback system provides the user with tactical feedback when the flexible housing 38 contacts an unseen or unexpected obstruction. This reduces the risk of the user damaging the camera control system or harming a patient during a surgical procedure by driving the camera 44 into the patient's body.

In some embodiments, the haptic feedback system may optionally include a visual or auditory indicator, such as light on the interior of the FPV headset 10 or a sound emitter mounted on the endoscope 20, which may be activated when the rotation threshold or pressure threshold is exceeded.

In certain embodiments, the haptic feedback system is differentially activated upon exceeding the rotation threshold and upon exceeding the pressure threshold. For example, the at least one vibrating component 68 may activate at a low frequency upon exceeding the rotation threshold and increase in intensity as rotation of the user's head increasingly exceeds the rotation threshold. The at least one vibrating component 68 may activate at a high frequency upon exceeding the pressure threshold and increase in intensity as pressure increases. If the pressure signal exceeds a second, higher pressure threshold, the visual or auditory indicator is also activated and the microcontroller 48 prevents any further movement of the camera 44 in the direction of the detected pressure signal.

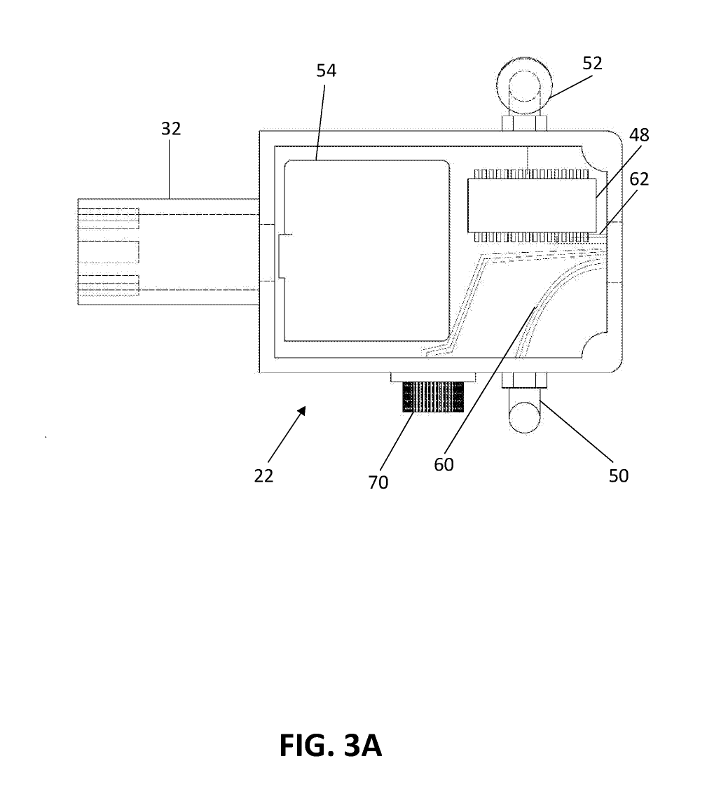

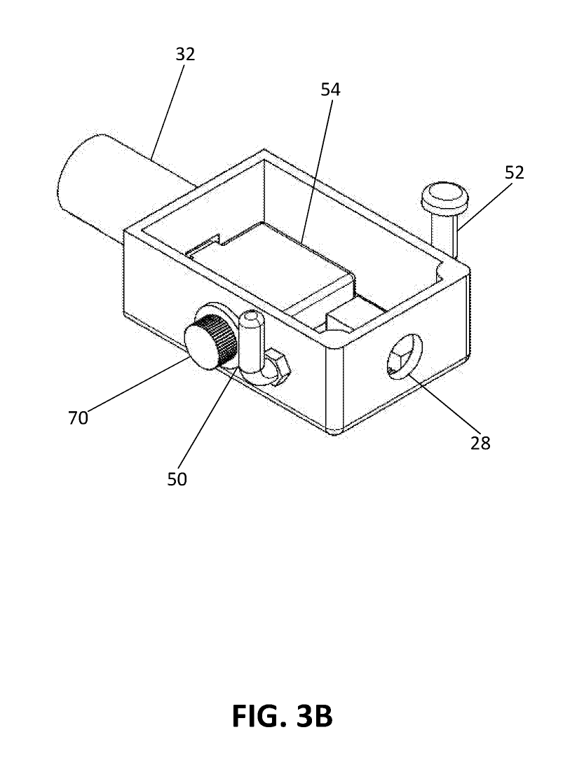

As shown in FIG. 5, the camera control system optionally includes a light source 46 mounted at least partially within the flexible housing 38. The light source 46 is configured to emit light in a direction substantially aligned with the field of view of the camera 44. The light source 46 is preferably a variable light source controllable by the user or other individual. In the depicted embodiment, as shown in FIGS. 2A, 2B, 3A and 3B, the intensity of light emitted by the variable light source 46 is controlled by a potentiometer 70 operatively coupled to the variable light source 46. In the embodiment shown in FIG. 5, the light source 46 is mounted underneath the lens(es) 72 of the camera 44, allowing the light source 46 to function as a spotlight and project in the camera's field of view. In other embodiments, the light source 46 may be a ring light surrounding the camera 44. In a further embodiment, the light source 46 may be completely within inside the flexible housing 38, which would require the flexible housing 38 to be transparent or translucent. In this embodiment, the light source 46 may be used to provide ambient light rather than projecting a beam of light.

In various embodiments, the mounting fixture 32, control housing 22, support member 26, actuator housing 24, and camera mount 34 are manufactured of any suitable rigid plastics or metals. Various embodiments include servomotors 56, 58 of varying size, make, speed and torque and include different methods of mounting the servomotors 56, 58 to the endoscope 20 and to the flexible housing 38 or camera 44. Various embodiments include alternative routes for wires or the replacement of the first or second electrical pathways 60, 62 with wireless systems.

The foregoing detailed description is given primarily for clearness of understanding and no unnecessary limitations are to be understood therefrom, for modifications can be made by those skilled in the art upon reading this disclosure and may be made without departing from the spirit of the invention. While this invention has been discussed in connection with imaging in conjunction with robotic surgery, it should be understood that this invention is suitable for use in any context, medical or non-medical, where it is advantageous for a user to see into difficult to view locations. Such locations may include the interior of a patient's thoracic cavity, the interior of an engine or other mechanical device, down a hole or pipe, or other locations as may be envisioned by the reader.

What is claimed is:

1) A camera control system, comprising:
a headset configured to be worn by a user, the headset including an orientation detection unit configured to transmit a control signal in response to detecting an orientation of the orientation detection unit; and
an endoscope including
a camera,
means for rotating the camera on a first axis,
means for rotating the camera on a second axis, and
a control unit configured to receive the control signal and control the means for rotating the camera on the first axis and the means for rotating the camera on the second axis.

2) The camera control system of claim 1, wherein the first axis and the second axis are not identical.

3) The camera control system of claim 1, further comprising a video transmitter in communication with the camera, wherein a video signal is transmitted from the camera to the video transmitter and from the video transmitter to the headset.

4) The camera control system of claim 3, wherein the headset includes means for displaying the video signal to the user.

5) The camera control system of claim 1, wherein the orientation detection unit includes a gyroscope and a control signal transmitter for transmitting the control signal to the control unit.

6) The camera control system of claim 1, wherein the orientation detection unit detects the orientation of the orientation detection unit on the first axis and the second axis, and wherein the control unit rotates the camera on the first axis and the second axis to match the orientation of the orientation detection unit.

7) The camera control system of claim 6, wherein the orientation detection unit detects the orientation of the orientation detection unit on a third axis, and wherein the control unit controls a zoom feature of the camera based on the orientation of the detection unit on the third axis.

8) The camera control system of claim 1, wherein the control unit is configured to control the means for rotating the camera on the first axis and the means for rotating the camera on the second axis when the control signal exceeds a predetermined rotation threshold.

9) The camera control system of claim 8, wherein the predetermined rotation threshold is two degrees of rotation in one of the first axis and the second axis.

10) The camera control system of claim 8, wherein the predetermined rotation threshold is five degrees of rotation in one of the first axis and the second axis.

11) The camera control system of claim 8, further comprising a haptic feedback system, wherein the haptic feedback system is activated when the control signal exceeds the predetermined rotation threshold.

12) The camera control system of claim 11, wherein the haptic feedback system includes at least one vibrating component attached to the headset.

13) The camera control system of claim 1, the endoscope includes a flexible housing, and wherein the camera located at least partially within the flexible housing.

14) The camera control system of claim 13, wherein the means for rotating the camera on the first axis is a first servomotor coupled to one of the flexible housing and the camera, whereby activation of the first servomotor deflects the camera along the first axis.

15) The camera control system of claim 13, wherein the means for rotating the camera on the second axis is a second servomotor coupled to one of the flexible housing and the camera, wherein activation of the second servomotor deflects the camera along the second axis.

16) The camera control system of claim 13, further comprising at least one pressure sensor mounted on the flexible housing.

17) The camera control system of claim 16, wherein the at least one pressure sensor is configured to output a pressure signal to the control unit.

18) The camera control system of claim 17, wherein the control unit controls the means for rotating the camera on the first axis and the means for rotating the camera on the second axis based on the control signal and the pressure signal.

19) The camera control system of claim 17, further comprising a haptic feedback system, wherein the haptic feedback system is activated when the pressure signal exceeds a predetermined pressure threshold.

20) The camera control system of claim 19, wherein the haptic feedback system includes at least one vibrating component attached to the headset.
